(19) United States
(12) Patent Application Publication
(10) Pub. No.: US 2016/0000550 A1
(43) Pub. Date: Jan. 7, 2016

(54) METHODS FOR TREATING DISEASES OF THE COLON

(71) Applicant: Deborah Nagle, Arlington, MA (US)

(72) Inventor: Deborah Nagle, Arlington, MA (US)

(21) Appl. No.: 14/324,178

(22) Filed: Jul. 5, 2014

(51) Int. Cl. A61F 2/04; A61F 2/82; A61B 17/068; A61B 17/00; A61F 2/00

(52) U.S. Cl. CPC A61F 2/04; A61B 17/00234; A61B 17/00491; A61F 2/0063; A61B 17/068; A61F 2/82; A61B 2017/00269; A61F 2250/0067; A61F 2002/045; A61F 2002/0072; A61F 2230/0069

(57) ABSTRACT

Methods for treating diseases of the rectum or colon are provided. The methods can comprise accessing the rectal mucosa through a trans-anal approach; performing a mucosectomy to remove mucosal tissue from at least a portion of the rectal or colonic wall; and securing a flexible scaffold material to a region of the rectal or colonic wall from which mucosa has been removed, wherein the scaffold material is selected to facilitate cellular ingrowth and formation of new mucosal tissue.

### RELATED APPLICATIONS

This application is related to a U.S. Provisional Application filed Jul. 5, 2013, and titled Method of Treating Ulcerative Colitis Without Colectomy; a U.S. Provisional Application filed Nov. 8, 2013, and titled Method of Treating Ulcerative Colitis Without Colectomy; and PCT Application Number PCT/US2014/033365 filed Apr. 8, 2014, each of which is incorporated herein by reference in its entirety.

### BACKGROUND

The present disclosure relates to methods for treating diseases of the colon, and more particularly, to methods of treating inflammatory bowel disease. Inflammatory Bowel Disease (IBD) consists or two independent diseases: Crohn's Disease (CD) and Ulcerative Colitis (UC), together affecting 833,000 Americans. While CD can manifest itself anywhere along the digestive tract and often affects the entire thickness of the digestive tract wall, UC is confined to the colon and rectum and affects only the mucosa (inner lining) of the wall. There is also an increased risk for colorectal cancer associated with those affected by IBD, thus giving rise to the need for treatment of both conditions. Medical treatments, including use of anti-inflammatories, which may have severe side effects, may fail in treating the symptoms of UC, and complete removal of the colon and the rectum (proctocolectomy) is the only known “cure” for UC when the patient is refractory to all other medical treatments. Proctocolectomy requires an ileostomy to facilitate waste removal.

Alternatives to first lines of treatment medications with potentially harmful side effects and second lines of treatment (proctocolectomy) are needed for treating and curing UC, for example. Artificial scaffolds useful for tissue regeneration have been developed, but have not been utilized to treat the colon. Purified collagen, gelatin, fat, hyaluronic acid, and synthetic materials have been used clinically in regenerative medicine for the treatment of urinary incontinence, reflux disease, laryngeal pathologies, and neonatal cardiomyocytes. Overly purified, chemically modified, or certain synthetic materials can lead to adverse immune responses by the host and limit cell migration into the matrix.

Naturally occurring extracellular matrix (ECM)-derived scaffolds possess many bioactive properties and have been used for the treatment of a variety of tissues, including lower urinary tract structures, esophagus, cardiac tissue, and musculo-skeletal structures. Many of these scaffolds are derived from non-human tissue. None has been described for use in the treatment or regeneration of lower intestine tissue, such as colonic tissue, and none has been specifically described for the treatment of ulcerative colitis.

### SUMMARY

The present application provides improved methods for treating the rectum and colon, including removal and regeneration of diseased muscosal tissues using processed ECM, synthetic scaffolds, and/or combinations of the two. According to certain embodiments, methods for treating certain conditions of the colon and rectum are provided. A preferred method can comprise accessing the rectal or colonic mucosa through a trans-anal approach; performing a mucosectomy to remove mucosal tissue from at least a portion of the rectal or colonic wall; and securing a flexible scaffold material to a region of the colonic wall from which mucosa has been removed, wherein the scaffold material is selected to facilitate cellular ingrowth and formation of new mucosal tissue.

According to certain embodiments, a method for treating the rectum or colon is provided. The method can comprise performing a mucosectomy to remove mucosal tissue from at least a portion of a rectal or colonic wall; and securing a flexible and elastic scaffold material to a region of the rectal or colonic wall from which mucosa has been removed, wherein the scaffold material is selected to facilitate cellular ingrowth and formation of new mucosal tissue.

According to certain embodiments, a device for treating the rectum or colon is provided. The device can comprise a porous scaffold having a tubular shape, wherein the scaffold material is selected to facilitate cellular ingrowth and formation of new mucosal tissue, and wherein the scaffold has sufficient elasticity and flexibility to withstand forces exerted by rectal or colonic peristalsis when implanted in the rectum or colon. The scaffold preferably has sufficient thickness to prevent collapse of the scaffold as a result of stricture or contraction.

The presently described methods and devices allow removal and regeneration of diseased rectal or colonic tissue to successfully treat conditions such as ulcerative colitis or other diseases affecting the mucosa. The methods include use of processed matrices, such as extracellular tissue matrices and/or synthetic materials that facilitate ingrowth of cells and mucosal formation.

The disclosed scaffolds, when used in accordance with the surgical methods described herein, can allow regeneration of mucosa while reducing the likelihood of various complications associated with mucosectomy or rectal or colonic surgery generally. For example, the disclosed methods can be performed using minimally invasive techniques, including a trans-anal mucosal resection and/or using endoscopic mucosal resection (EMR), which can involve insertion of a fluid under the mucosa to protect the underlying tissue prior to excision. Treatment of conditions in the colon that are proximal to the rectum can employ an endoscope having a camera and light source to visualize the surgical site, one or more working channels through which tools can be inserted to cut and remove tissue, and is steerable to navigate through the angular bends in the colon. Furthermore, the scaffold materials can be inserted using a catheter for placement and enable successful mucosal growth, which decreases the likelihood of stricture or scar formation within the surgical site. The catheter can include a radially mounted balloon with a stent and ECM mounted thereon during delivery. Radioopaque elements on the stent can assist in visualization for correct placement when the balloon is expanded for deployment. Bioabsorbable staples operate as attachment elements, which temporally hold the stent in place. The peristaltic movement of the bowel will naturally remove the stent after sufficient ingrowth.

Furthermore, treatment of rectal or colonic conditions requiring regeneration of tissue or formation of new tissue can be particularly complicated due to dynamic peristaltic movements in the gastrointestinal tract. For example, peristalsis can subject treatment sites to varying stresses throughout recovery, and such stresses can cause treatment devices to loosen, migrate, or tear. The presently discloses methods provide for scaffold materials with flexibility and elasticity, such as conformable tubes, for example, thereby allowing the materials to withstand peristaltic movements while allowing for tissue ingrowth.

### DESCRIPTION OF THE DRAWINGS

FIG. 1 is a perspective partial cut-away view of a the rectum and colon.

FIG. 2 illustrates a process for trans-anal resection of rectal or colonic mucosa, according to certain embodiments of the present application.

FIG. 3 illustrates a perspective cut-away view of a portion of the rectum and colon after mucosectomy.

FIG. 4 illustrates a perspective cut-away view of a portion of the rectum and colon after rectal mucosectomy and during further mucosal removal via endoscopic mucosal resection.

FIG. 5 illustrates a perspective cut-away view of a portion of the rectum and colon after mucosectomy.

FIG. 6A illustrates a scaffold material for treating the rectum or colon, according to various embodiments.

FIG. 6B illustrates a scaffold material for treating the rectum or colon, according to various embodiments.

FIG. 6C illustrates a scaffold material for treating the rectum or colon, according to various embodiments.

FIG. 7 illustrates a stent for securing scaffold materials adjacent to rectal or colonic tissue after mucosectomy.

FIG. 8 illustrates a perspective cut-away view of a portion of the rectum and colon after mucosectomy and placement of a scaffold material to facilitate mucosa regeneration.

FIG. 9 illustrates a perspective cut-away view of a portion of the rectum and colon after mucosectomy and placement of two scaffold materials to facilitate mucosa regeneration.

FIG. 10A illustrates a perspective cut-away view of a portion of the rectum and colon after mucosectomy and placement of a scaffold material and stent to facilitate mucosa regeneration.

FIG. 10B illustrates a perspective cut-away view of a portion of the rectum and colon after mucosectomy and placement of two scaffold materials and a single stent to facilitate mucosa regeneration.

FIG. 11A is a hematoxylin and eosin (H&E) section of a distal portion (identified in FIG. 11B) of dog rectum twelve weeks post-operative after mucosectomy and treatment with small intestine submucosal tissue matrix, as described in Example 1.

FIG. 11B is a distal portion of dog rectum twelve weeks post-operative after mucosectomy and treatment with small intestine submucosal tissue matrix, as described in Example 1.

FIG. 12A is an H&E section of a distal portion (identified in FIG. 12B) of dog rectum twelve weeks post-operative after mucosectomy and treatment with small intestine submucosal tissue matrix, as described in Example 1.

FIG. 12B is a distal portion of dog rectum twelve weeks post-operative after mucosectomy and treatment with small intestine submucosal tissue matrix, as described in Example 1.

FIG. 13A is an H&E section of a distal portion (identified in FIG. 13B) of dog rectum twelve weeks post-operative after mucosectomy and treatment with small intestine submucosal tissue matrix, as described in Example 1.

FIG. 13B is a distal portion of dog rectum twelve weeks post-operative after mucosectomy and treatment with small intestine submucosal tissue matrix, as described in Example 1.

FIG. 14A is an H&E section of a distal portion (identified in FIG. 14B) of dog rectum twelve weeks post-operative after mucosectomy and treatment with small intestine submucosal tissue matrix, as described in Example 1.

FIG. 14B is a distal portion of dog rectum twelve weeks post-operative after mucosectomy and treatment with small intestine submucosal tissue matrix, as described in Example 1.

FIG. 15 is a flow chart illustrating methods for treating the rectum or colon according to various embodiments.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

As used herein, the term “scaffold” refers to any biologic, synthetic, or hybrid materials having a structure and biologic properties that permit ingrowth of cells and regeneration of tissue or formation of new tissue. Scaffolds can include extracellular tissue matrices, synthetic matrices, and/or hybrid materials. As discussed below, suitable scaffolds can be in the form of a sheet or layered cylindrical device, or scaffolds can include gels or pastes. Suitable scaffolds can be acellular, or can be seeded with cells, e.g., stem cells.

As noted above, IBD comprises two distinct diseases, including ulcerative colitis (UC) and Crohn's disease (CD). Ulcerative colitis typically affects the distal colon and rectum and extends more proximally in a continuous fashion, while Crohn's disease may be characterized by skip lesions that can affect the entire gastrointestinal tract.

The present disclosure provides methods for treating the rectum or colon. The method can comprise accessing the rectal or colonic mucosa through a trans-anal approach; performing a mucosectomy to remove mucosal tissue from at least a portion of the rectal or colonic wall; and securing a flexible scaffold material to a region of the rectal or colonic wall from which mucosa has been removed, wherein the scaffold material is selected to facilitate cellular ingrowth and formation of new mucosal tissue.

Use of a trans-anal approach may be particularly suitable for treating ulcerative colitis. For example, for patients whose disease process primarily affects the rectum or recto-sigmoid colon, a trans-anal approach may be sufficient for removal of all or a significant portion of diseased mucosa. Such an approach can therefore eliminate the need for continuous medical therapy and can prevent the need for more invasive procedures, thus reducing recovery time and patient morbidity

According to certain embodiments a method for treating the rectum or colon is provided. The method can comprise performing a mucosectomy to remove mucosal tissue from at least a portion of a rectal or colonic wall; and securing a flexible and elastic scaffold material to a region of the rectal or colonic wall from which mucosa has been removed, wherein the scaffold material is selected to facilitate cellular ingrowth and formation of new mucosal tissue.

As discussed further below, the gastro-intestinal tract, including the rectum and colon, is characterized by ongoing peristaltic movement. Accordingly, any device that is implanted within and/or secured to rectal or colonic tissue will be subjected to repeated stretching and movement. Accordingly, in order to successfully treat rectal or colonic tissue and regenerate or produce new mucosa, specialized materials are needed. The materials should include structural and biologic characteristics that allow ingrowth of cells, formation of new tissue, prevent scar and stricture formation, and can withstand the mechanical demands without tearing or dislodging.

According to certain embodiments, a device for treating the rectum or colon is provided. The device can comprise a porous scaffold having a tubular shape, wherein the scaffold material is selected to facilitate cellular ingrowth and formation of new mucosal tissue, and wherein the scaffold has sufficient elasticity and flexibility to withstand forces exerted by rectal or colonic peristalsis when implanted in the rectum or colon.

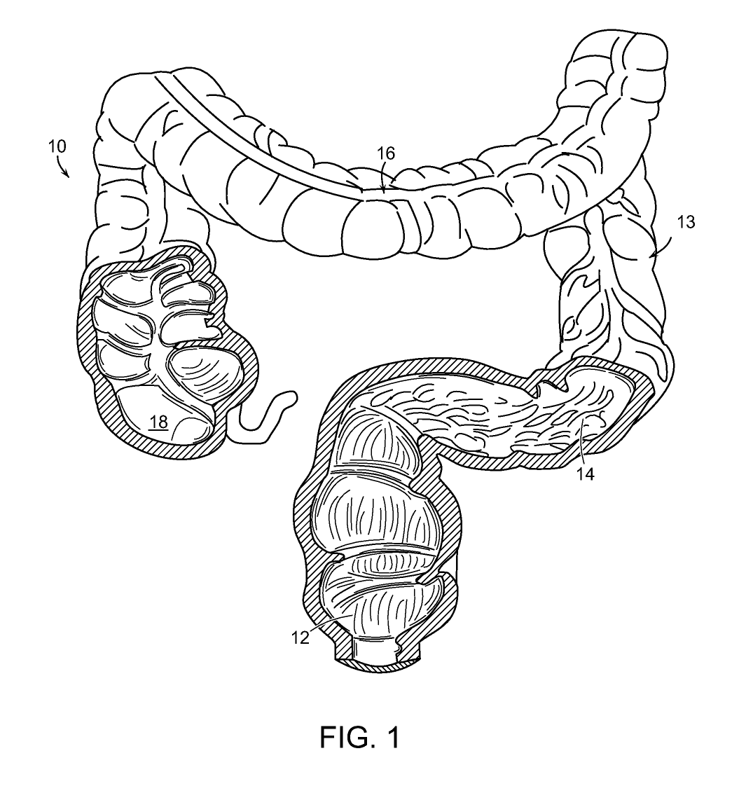

FIG. 1 is a perspective partial cut-away view of a colon 10. As shown, the colon 10 include the ascending colon 18, transverse colon 16, descending colon 22, sigmoid colon 14, and rectum 12. As noted above, UC is characterized by a process that begins in the rectum 12, and extends more proximally. Although UC can affect the entire colon 10, it is typically limited to more distal portions of the colon, thus making a trans-anal mucosal treatment desirable. As used herein, “colon” is to be understood to any portion of the colon, as identified in FIG. 1, including the rectum. As such, although the rectum and colon may be identified separately herein, the methods for removal of colonic mucosa will be understood to apply to procedures that are directly toward treatment of rectal mucosa, include treatment of rectal mucosa and other portions of the colon, or include treatment at sites proximal to the rectum, e.g., sigmoid colon.

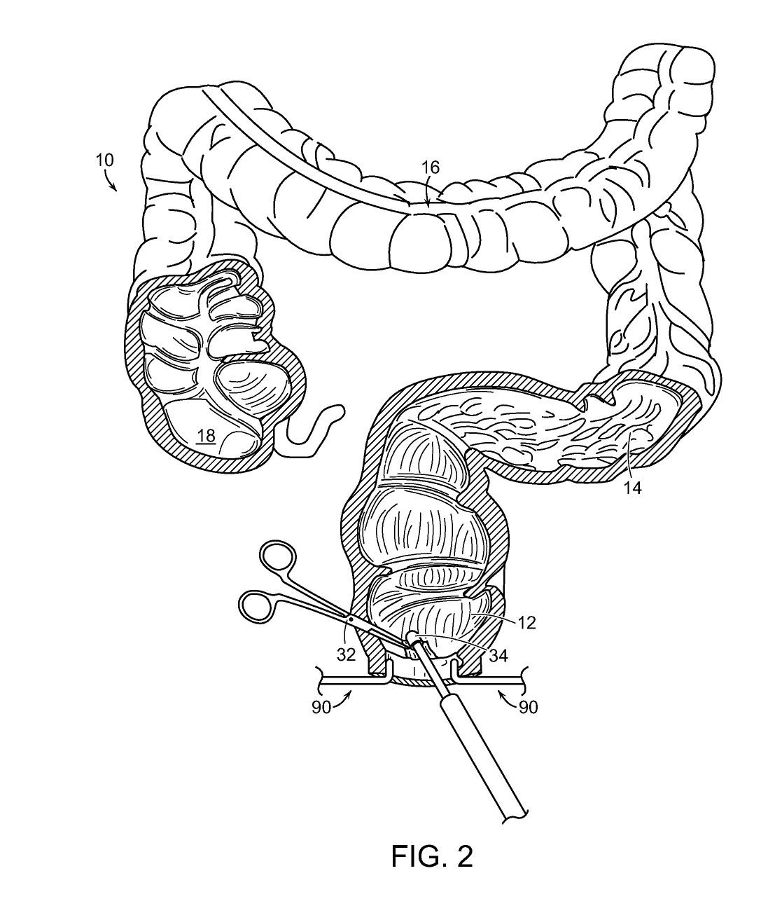

As noted, the methods of the present disclosure can include removal of mucosal tissue in the rectum 12, or more proximally. FIG. 2 illustrates a process for trans-anal resection of rectal or colonic mucosa, according to certain embodiments of the present application. As shown, the desired treatment site can be exposed using anal retractors 90, and mucosal resection can be performed using standard surgical instruments such as forceps 32, electro-cautery instruments 34, and other suitable instruments, e.g., blunt dissectors.

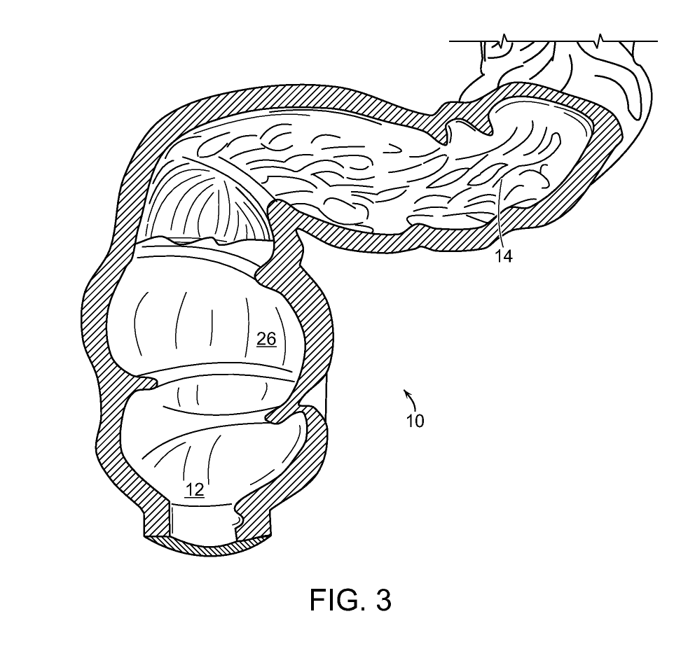

Using the trans-anal approach, a surgeon can select a beginning and end point to delimit the desired area for mucosal removal. The surgeon can then remove the target tissue circumferentially thereby leaving a region of rectum or recto-signmoid colon free from mucosal tissue, as illustrate in FIG. 3, which provides a perspective cut-away view of a portion of the colon 26 after mucosectomy.

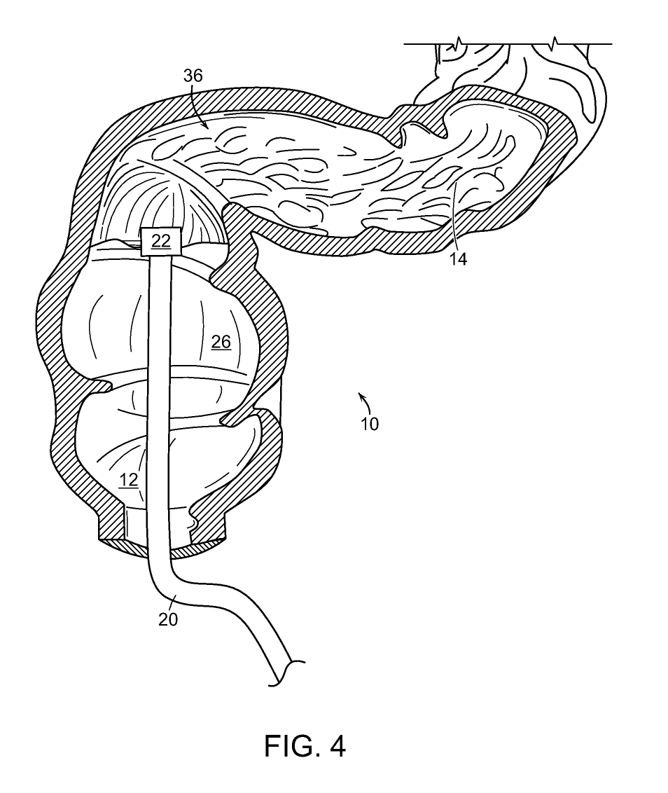

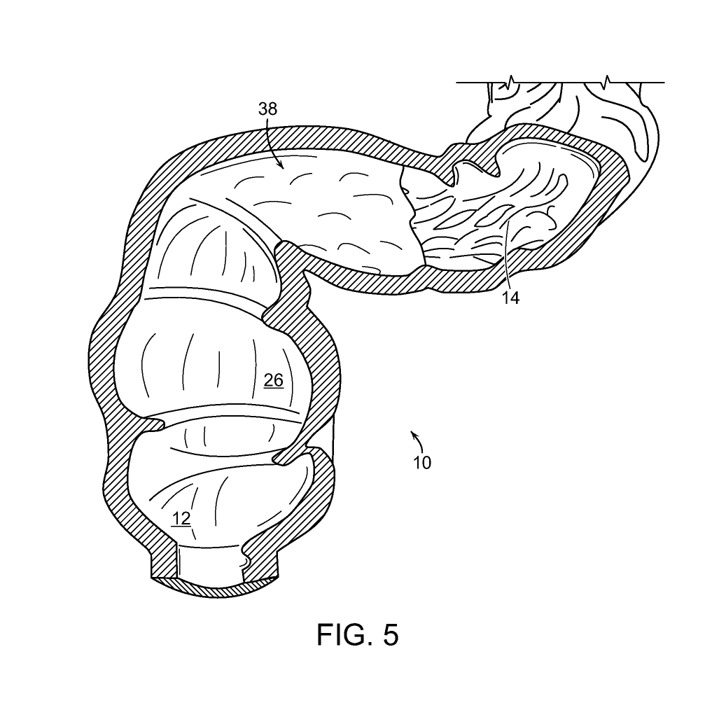

In certain cases, it may be desirable to remove addition mucosal tissue that cannot be accessed without additional instruments. Accordingly, in some embodiments, in order to remove tissue more proximally within the sigmoid colon or anywhere else in the colon, endoscopic mucosal resection (EMR) may be employed. For example, FIG. 4 illustrates a perspective cut-away view of a portion of the colon 26 after mucosectomy and undergoing further mucosal removal via EMR. As shown, EMR employs a scope 20 with specialized tools 22 configure to allow removal of mucosal tissue 36 at any colonic site. And FIG. 5 illustrates a perspective cut-away view of a portion of the colon after mucosectomy in the rectum 26 and in a portion of the sigmoid colon 36.

It will be appreciated that although the previous discussion contemplates mucosectomy in the rectum followed by EMR in the signmoid colon, the steps can be reversed such the EMR is performed first. Furthermore, if desired, EMR may be used for rectal mucosectomy.

After removal of mucosa in a desired area, a scaffold material can be secured to the treatment site. In general, the scaffold material is a flexible sheet, which may be in the form of a conduit or cylinder, which has biologic properties to allow cellular ingrowth and tissue regeneration. Specific examples of suitable scaffolds are discussed in more detail below, but generally, suitable scaffolds can include collagen-based materials, e.g., extracellular matrices (ECM), synthetic materials, and/or hybrid ECM-synthetic constructs. Further, the materials should be selected to have sufficient elasticity and flexibility to withstand movements due to peristalsis during the applicable recovery period.

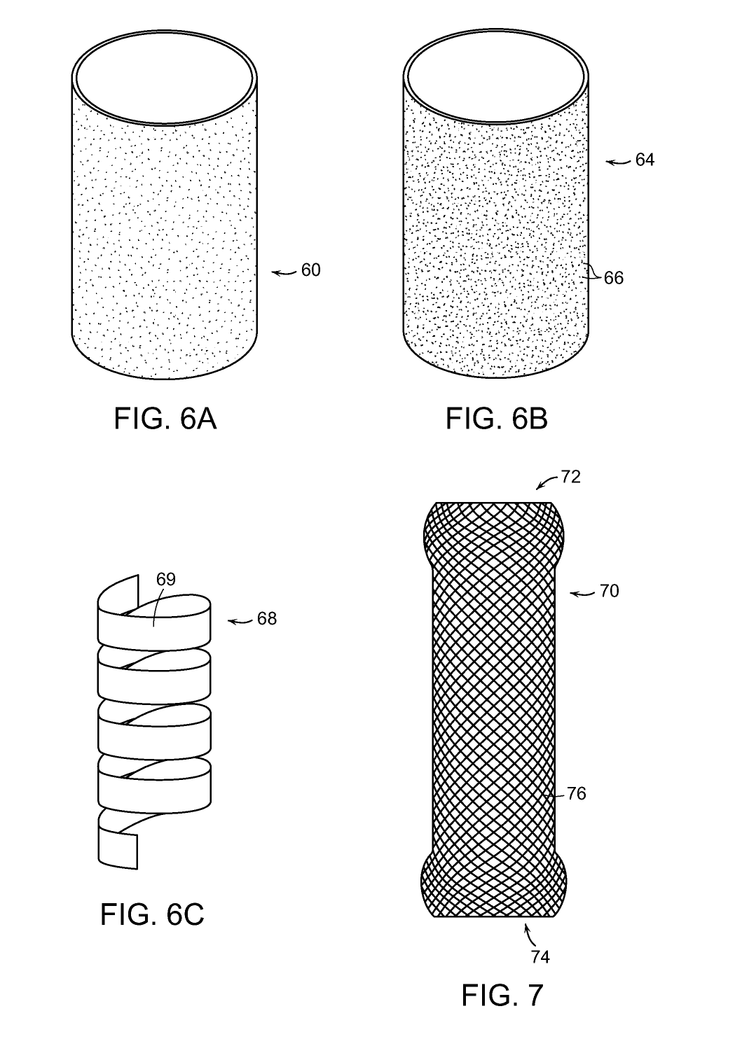

FIGS. 6A-6C illustrate various embodiments of scaffold materials for treating the rectum or colon. Each of the devices 60, 64, 68 can be formed from any of the materials discussed herein. Further, the devices 60, 64, 68 can have a variety of shapes or configurations. For example, each of the devices has a substantially cylindrical or tubular shape, but may be in the form of a flat, flexible sheet, which the surgeon may mold to the treatment site. Furthermore, the devices may be modified in various ways to increase conformability or elasticity. For example, the device 64 of FIG. 6B can include a material like that of FIG. 6A, but may include perforations 66, slits, or a meshed structure, as may be formed using known skin meshing devices, such as a ZIMMER® SKIN GRAFT MESHER. In other embodiments, the devices can be formed of a wound construct formed by an elongate sheet 69, as shown in FIG. 6C, and the wound construct can be expanded to contact the rectal or colonic wall.

In various embodiments, the devices and methods can include multiple layers of scaffold to allow more complete tissue coverage and/or to increase mechanical support. For example, the device can include at least two, at least four, at least eight, at least ten, or more layers. The layers may be laminated in a single device or may be formed during surgery by implanting multiple devices.

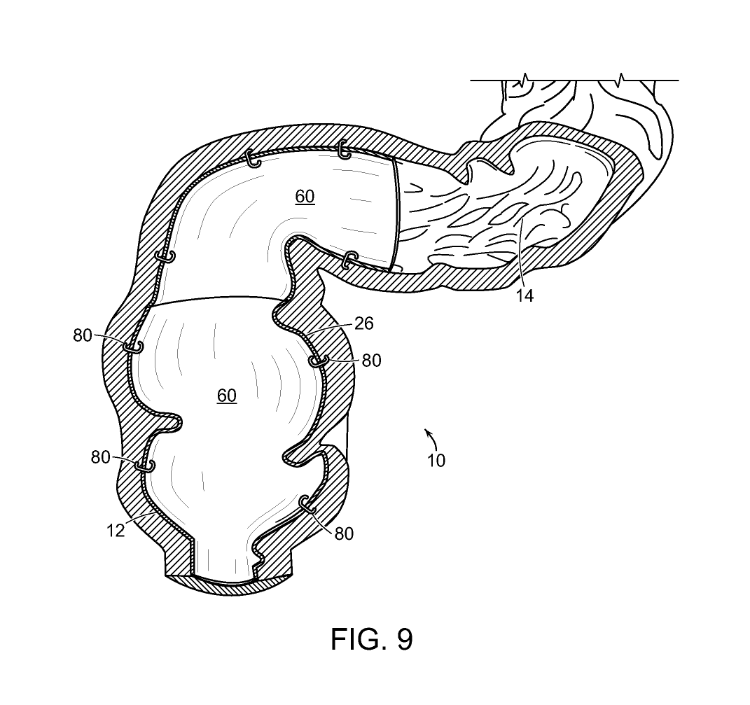

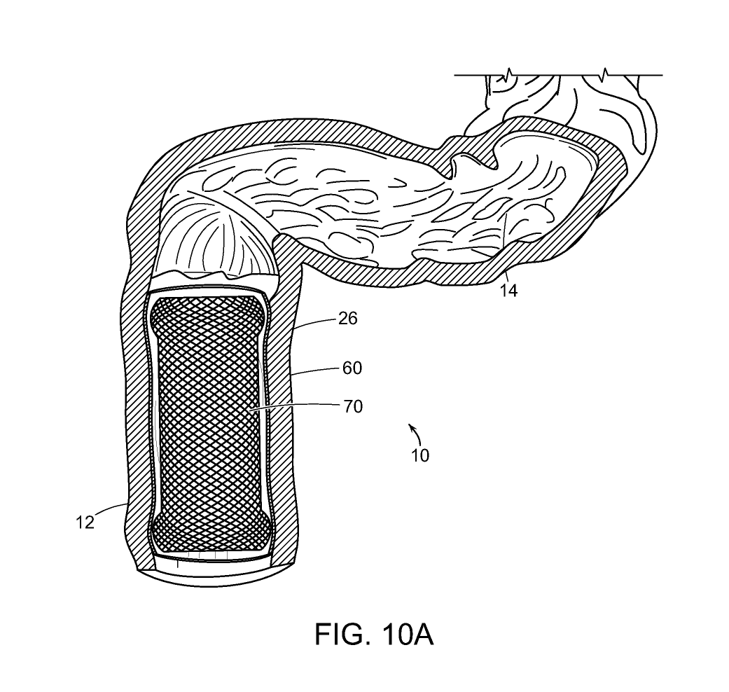

After placement of the scaffold in the region of the rectum or colon to be treated, the scaffold will be secured to the rectal or colonic wall. The scaffold can be secured using a variety of devices and techniques including, suturing, stapling, stenting, clipping, applying an adhesive (e.g., fibrin glue), or combinations thereof. FIG. 8 illustrates a perspective cut-away view of a portion of the rectum and colon after mucosectomy and placement of a scaffold material 60 to facilitate mucosa regeneration, and FIG. 9 illustrates a perspective cut-away view of a portion of the rectum and colon after mucosectomy and placement of two scaffold materials 60 to facilitate mucosa regeneration. As shown, in FIGS. 8 and 9, scaffold materials 60 are placed in the desired locations and secured in place using fasteners 80, which can include sutures, staples, or clips. Suitable fasteners should be selected so that they do not perforate the colon wall, e.g., have a depth of less than approximately 5 mm.

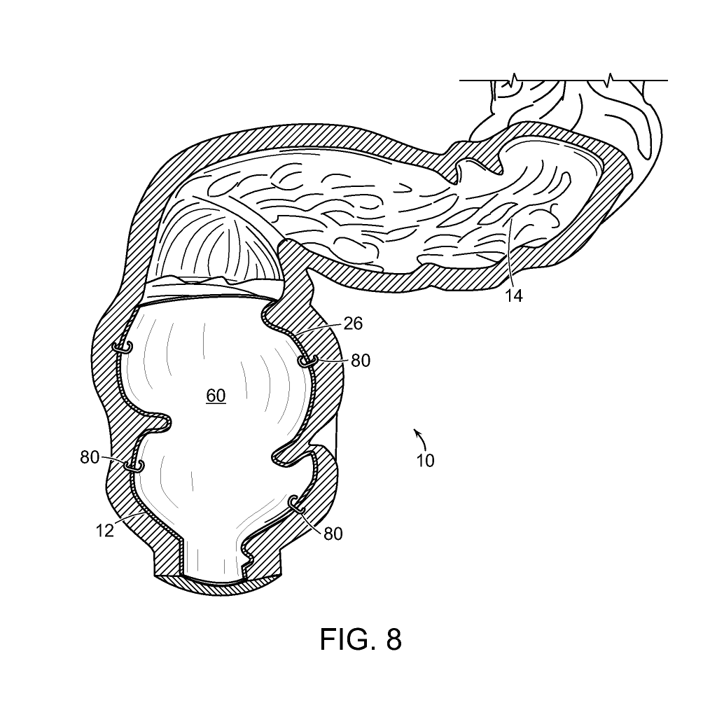

In some embodiments, a stent may be used to assist in securing a scaffold material in a portion of a colon. For example, FIG. 7 illustrates a colonic stent 70 for securing scaffold materials adjacent to colon tissue after mucosectomy. Gastrointestinal and colonic stents are known, and suitable stents can be selected based on size, patient anatomy, and/or ability to maintain position and be removed with desired. Certain commercial colonic stents include, for example, the EVOLUTION® COLONIC STENT (COOK MEDICAL), or the WALLFLEX® COLONIC STENT (BOSTON SCIENTIFIC CORPORATION), which are self-expanding metallic stents. However, as these stents are designed to hold open strictures, these would not be suitable for many patient situations, and require modification to a larger diameter and configured to avoid impingement on the anal canal for use in the rectum.

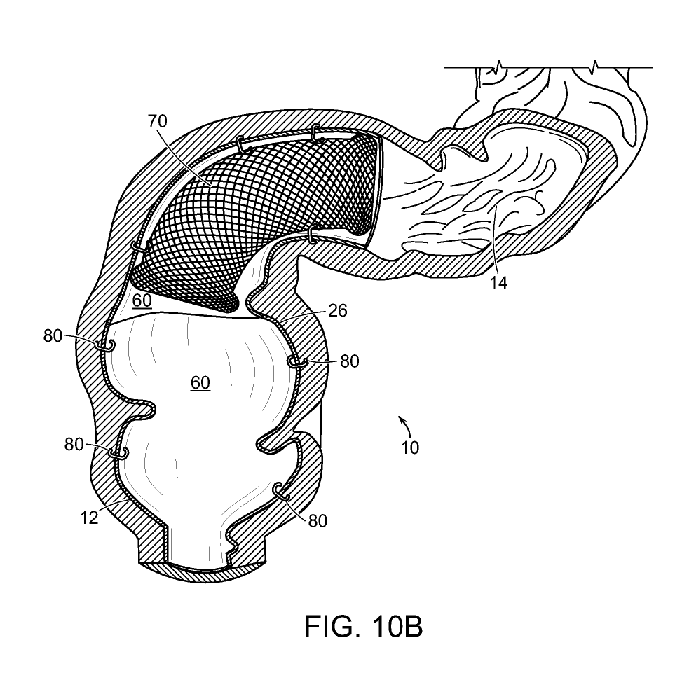

FIG. 10A illustrates a perspective cut-away view of a portion of the rectum and colon after mucosectomy and placement of a scaffold material 60 and stent 70 to facilitate mucosa regeneration, and FIG. 10B illustrates a perspective cut-away view of a portion of the rectum and colon after mucosectomy and placement of two scaffold materials 60 and a single stent 70 to facilitate mucosa regeneration. As shown, the stent can be used for placement of a scaffold in the rectum (FIG. 10A) and/or for placement of a scaffold in the sigmoid colon (FIG. 10B). Furthermore, as shown in FIG. 10B, a stent 70 can be used for placement of a more proximally located scaffold in the sigmoid colon 14, while fasteners 80 can be used to secure the scaffold 60 in the rectum. Furthermore, the stent 70 may be secured in position using clips, sutures, or other fasteners to prevent migration until a desired removal time.

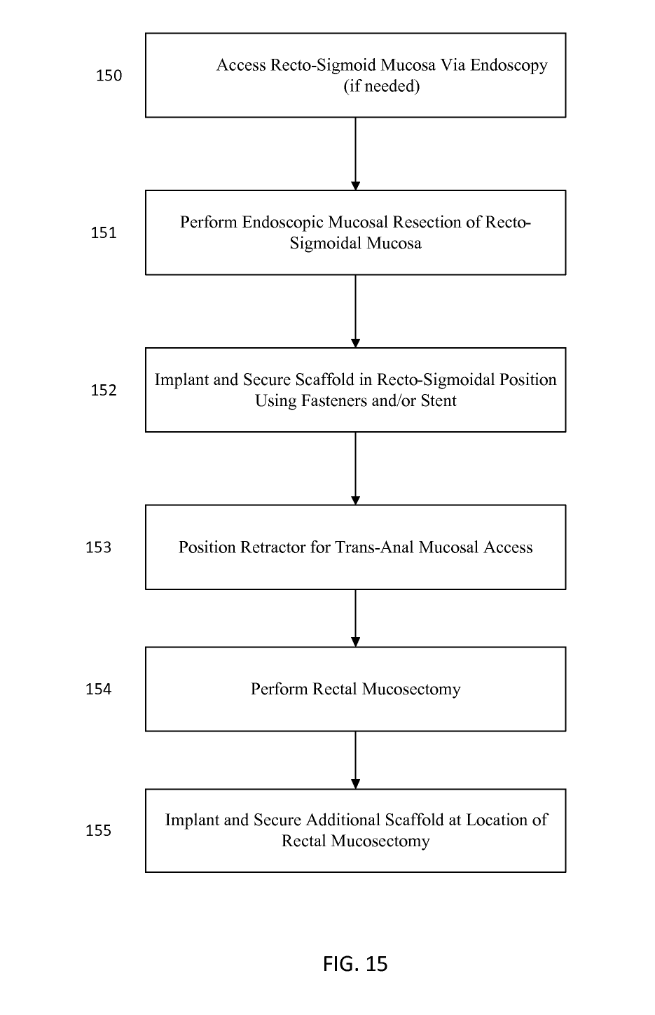

Exemplary methods for treating the rectum or colon are now described with respect to FIG. 15, which is a flow chart illustrating methods for treating the rectum or colon. As shown, the steps are described for treatment of sections of the rectum and sigmoid colon, and although described using one particular order of steps, it will be appreciated that the various steps may be performed in different orders depending on surgeon preference and/or patient specific factors.

As shown in FIG. 15, the procedure may begin by accessing more proximal sections of the colon (e.g., recto-sigmoid colon or sigmoid colon) via endoscopy (Step 150). And subsequently, endoscopic mucosal resection (EMR) may be performed in order to remove mucosa from the selected colonic section (Step 151). Next, a scaffold material, as described previously, can be implanted and secured to the tissue where mucosectomy has been performed (Step 152) using sutures, other fasteners, a stent, or combinations of sutures, fasteners, and a stent.

Next, a trans-anal mucosal resection may be performed to remove more distally located mucosa. Accordingly, an anal retractor can be position (Step 153) to assist in accessing the mucosa, and a trans-anal mucosectomy can be performed (Step 154). Accordingly, by performing the more proximal mucosectomy prior to the distal mucosectomy, exposed submucosal tissue in the distal colon may be less likely to be adversely affected by instrumentation used for treatment of the proximal sections. Finally, after performing the distal mucosectomy, an additional scaffold material is implanted and secured to the exposed submucosal tissue (Step 155).

### Example 1

An canine study was performed to test the feasibility of mucosal resection and regeneration. Operations were performed on four adult canines. Briefly, for each dog, a trans-anal mucosal resection was performed to circumferentially remove approximately four centimeters of rectal mucosa. Each canine was then treated by implantation of multi-laminate small intestine submucosal ECM (SIS-ECM) scaffolds having two, four, six, or eight layers. THE SIS-ECM was anchored in place with sutures. For the adult human colon, six or more layers of this material is preferred to provide the required support needed to prevent collapse or formation of strictures.

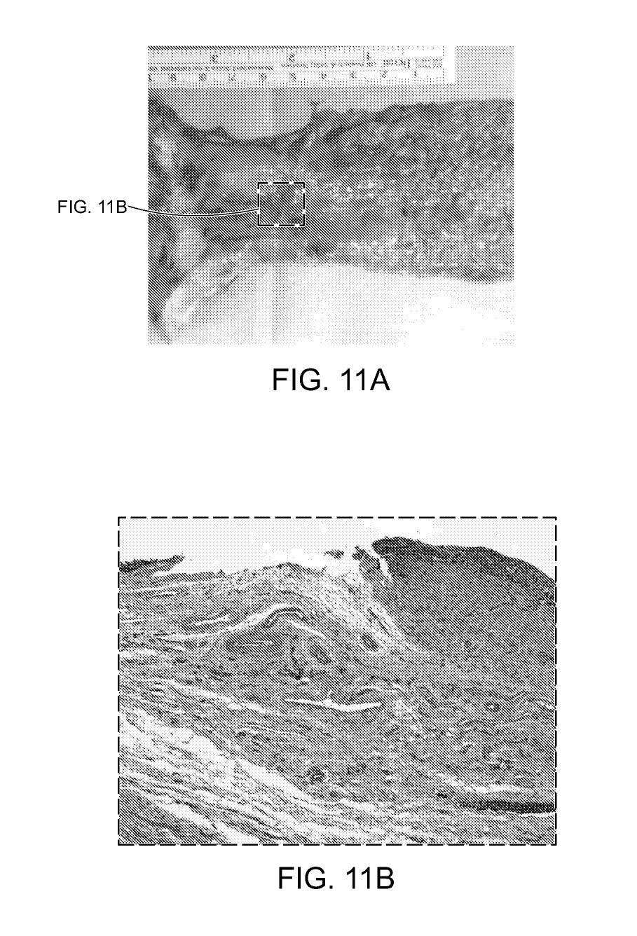

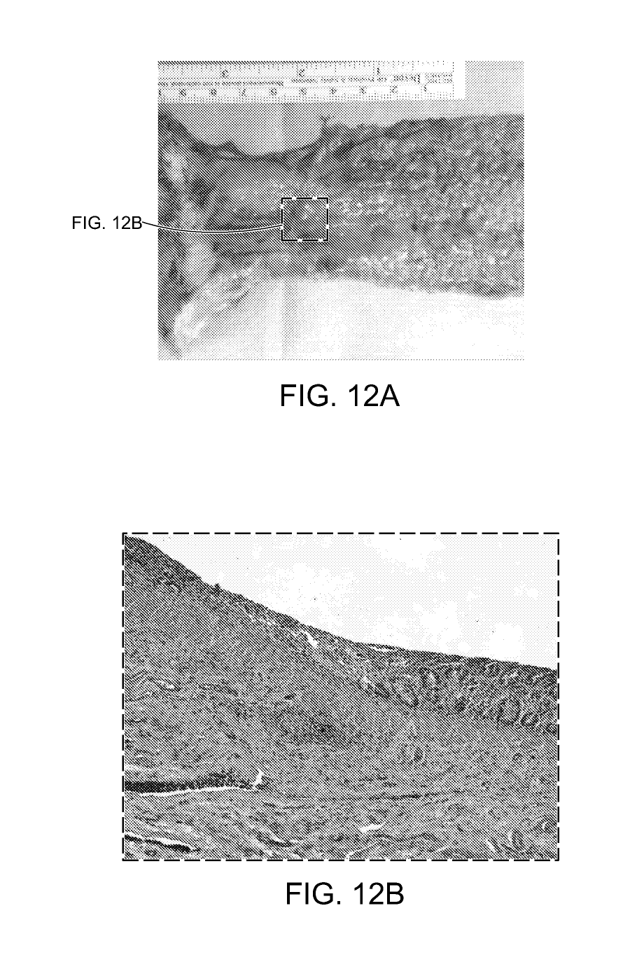

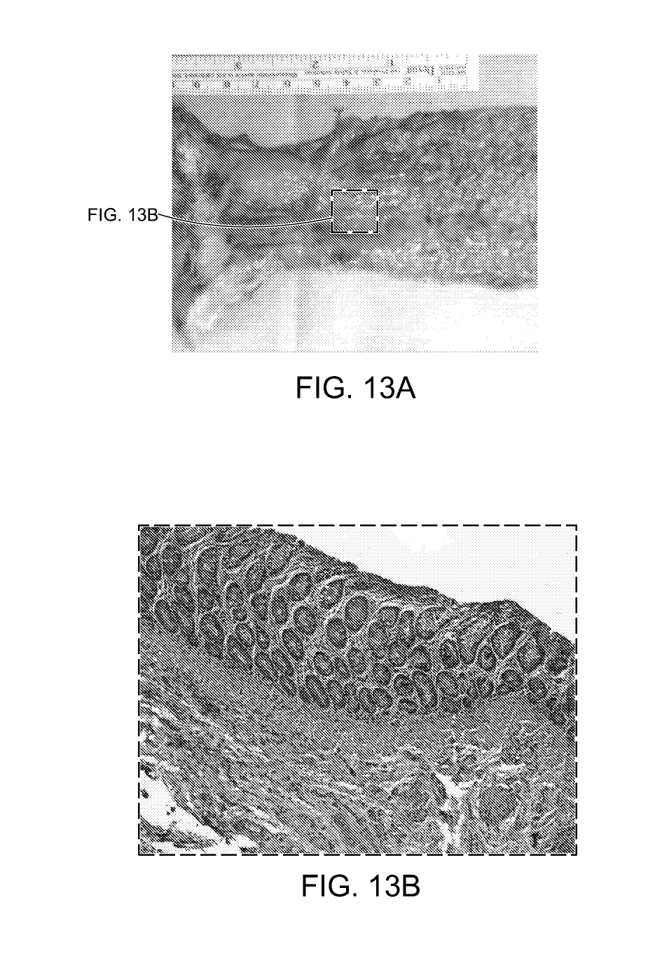

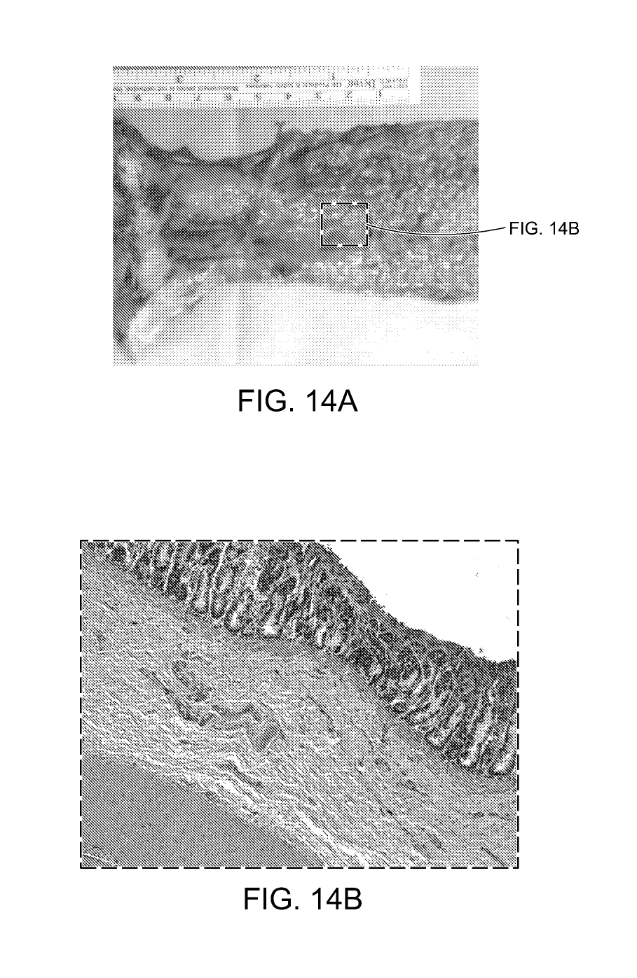

At twelve weeks, the animals were euthanized, and tissue samples were obtained. FIGS. 11A, 12A, 13A, and 14A are H&E sections of a distal portion (as indicated in FIGS. 11B, 12B, 13B, and 14B) of dog colon twelve weeks post-operative after mucosectomy and treatment with small intestine submucosal tissue matrix. As shown, in FIGS. 12A, 13A, and 14A, mucosa in more proximate colon was completely regenerated, but incomplete mucosal coverage was noted in the distal colon (FIG. 11A) which can occur do to incomplete ingrowth during the period prior to removal for examination.

In all animals, the new mucosa appeared grossly and microscopically normal. In addition, increasing the number of layers of SIS-ECM resulted in increased mucosal coverage, and no signs of stricture were found in any of the animals. Accordingly, trans-anal mucosectomy and placement of regenerative scaffolds such as SIS-ECM appears capable of providing mucosal regeneration without formation of excessive scar or stricture.

### Exemplary Scaffold Materials

A variety of suitable scaffold materials may be used according to the methods of the present disclosure. For example, any suitable matrix material that permits cellular ingrowth and tissue regeneration may be selected. Such materials can include processed extracellular matrix materials, synthetic materials, reconstituted collagen, or materials including combinations of ECM, synthetics, or collagen-based materials. Self-assembling peptides such as those pioneered by Zhang et al. can also be used to fabricate suitable matrix scaffold assemblies or gels for implantation in the colon.

Suitable SIS-ECM materials are described for example by Badylak, S. F., et al., Esophageal Reconstruction with ECM and Muscle Tissue in a Dog Model, Journal of Surgical Research 128, 87-97 (2005); Badylak, S. F. et al., Esophageal Preservation in Five Male Patients After Endoscopic Inner-Layer Circumferential Resection in the Setting of Superficial Cancer: A Regenerative Medicine Approach with a Biologic Scaffold, TISSUE ENGINEERING: Part A, Volume 17, Numbers 7 and 8, 2011; Neoponice, A., An extracellular matrix scaffold for esophageal stricture prevention after circumferential EMR, GASTROINTESTINAL ENDOSCOPY Volume 69, No. 2: 2009; Badylak et al., “Extracellular Matrix as a Biological Scaffold Material: Structure and Function,” Acta Biomaterialia (2008), doi:10.1016/j.actbio.2008.09.013; and T. Hoppo, et al., A Novel Esophageal-preserving Approach to Treat High-grade Dysplasia and Superficial Adenocarcinoma in the Presence of Chronic Gastroesophageal Reflux Disease, World J Surg., 27 Jun. 2012. Each of these are hereby incorporated by reference, in their entirety.

As discussed above, suitable materials may be in the form of a sheet or cylinder, and may be configured to expand outward when deployed within the colon. A number of commercially available tissue matrix products, including acellular tissue matrices in which all native cells have been removed, may be used, including for example, TISSUEMEND®, SURGIMEND®, CONNEXA®, ALLODERM®, STRATTICE®. See Cornwell, K G et al, “Extracellular matrix biomaterials for soft tissue repair,” Clin. Podiatry Med Surg, 26:507-523 (2006). However, these materials may require modification, such as the adding of a further support layer or increased thickness to have the support required to enable ingrowth over the required time periods within the rectum or colon.

Furthermore, ECM materials may be formed from a number of tissue sources, including, for example SIS, tendon, urinary bladder, skeletal muscle, esophagus, large intestine, small intestine, gastric tissue, pericardial tissue, dermis, ligament, muscle, adipose tissue, and fascia. In addition, suitable materials may be formed from human (allograft) or non-human (xenograft) sources. For example, suitable non-human sources may include pigs, cows, horses, canine, or other animals. In addition, for non-human tissues, further processing may be performed to remove antigenic components. See Xu, Hui. et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15, 1-13 (2009), which is incorporated by reference in its entirety.

In addition, in some embodiments, the scaffold can comprise a synthetic material and/or a composite formed of a biologic materials such as ECM and a synthetic. For example, one composite material comprising porcine extracellular dermis and poly(ester urethane)urea (PEUU) is describe by Hong, Y. et al, “An elastomeric patch electrospun from a blended solution of dermal extracellular matrix and biodegradable polyurethane for rat abdominal wall repair,” Tissue Engineering, Part C 18(2): 122-132. This composite material may be useful as the PEUU synthetic component provides improved elasticity, flexibility, and mechanical support. In addition, a variety of other synthetic ECM-like materials are available and may be employed along with the disclosed surgical techniques. See Alberti C. et al., “About recent developments of synthetic polymers for a suitable cell adhesion/growth support in tissue engineering-based either augmentation cystoplasty or neobladder,” Ann Ital Chir. June 23:85, pii: S0003469X14022842. (2014); see also Schulte V A et al., “Hydrogel-fibre composites with independent control over cell adhesion to gel and fibres as an integral approach towards a biomimetic artificial ECM,” Biofabrication, 6(2):024106. (2014) doi: 10.1088/1758-5082/6/2/024106.

The preparation of SIS extracellular tissue matrices from a segment of small intestine is detailed In U.S. Pat. No. 4,902,508, U.S. Pat. No. 5,215,826, and U.S. Pat. No. 5,514,533, the disclosures of which are expressly incorporated herein by reference. A segment of intestine is first subjected to abrasion using a longitudinal wiping motion to remove both the outer layers (particularly the tunica serosa and the tunica muscularis) and the inner layers (the luminal portions of the tunica mucosa). Typically, the SIS is rinsed with saline and optionally stored in a hydrated or dehydrated state until use as described below.

In many cases, the scaffold materials can comprise a layer of thin and flexible materials such as a sheet, or conformable tubes, as illustrated in FIGS. 6A-C. In some embodiments, the material includes multiple layers e.g., at least 2, 4, 6, 8, 10, or more layers, which may be selected based on patient indications, to ensure complete coverage, and/or to provide mechanical support. Furthermore, as discussed below, suitable scaffolds may also be in the form of a suspension, gel, or paste, which can be applied directly to mucosal tissue.

In various embodiments, the devices and methods disclosed herein can further comprise addition of one or more therapeutic agents. For example, in various embodiments, in order to treat or prevent infection, reduce scar formation, or for other purpose, the devices can be coated with or impregnated with one or more of antibiotics, antimicrobials, antifungals, anti-inflammatories, tissue growth factors, and ionic silver.

In addition, in order to facilitate more rapid mucosal formation, the scaffolds may be treated with cells, such as mesenchymal stem cells. Methods for treating the colon, including methods for treating IBD with stem cells are described, for example, in Lanzoni, G. et al, “Inflammatory bowel disease: Moving toward a stem cell-based therapy,” World J Gastroenterol. Aug. 7, 2008; 14(29): 4616-4626.

### Solutions of SIS

The present fluidized compositions are prepared as solutions or suspensions of intestinal submucosa by comminuting and/or digesting the submucosa with a protease, such as trypsin or pepsin, for a period of time sufficient to solubilize said tissue and form a substantially homogeneous solution. The intestinal submucosa starting material is comminuted by tearing, cutting, grinding, shearing and the like. Grinding the submucosa in a frozen or freeze-dried state is preferred although good results can be obtained as well by subjecting a suspension of pieces of the submucosa to treatment in a high speed, high shear blender and dewatering, if necessary, by centrifuging and decanting excess water. The comminuted intestinal submucosa can be dried to form submucosa powder. Thereafter, it can be hydrated, that is, combined with water or buffered saline and optionally other pharmaceutically acceptable excipients to form a tissue graft composition as a fluid having a viscosity of about 2 to about 300,000 cps at 25° C. The higher viscosity graft compositions can have a gel- or paste-like consistency. The present compositions can be sterilized using art-recognized sterilization techniques such as exposure to ionizing radiation.

### SIS Suspensions

SIS specimens prepared as described above are minced or chopped into arbitrarily small pieces using tissue scissors, a single-edged razor blade, or other appropriate cutting implement. The specimens are placed in a flat bottom stainless steel container, and liquid nitrogen is introduced into the container to freeze the specimens to prepare them for comminuting.

The frozen SIS specimens are then comminuted to form a coarse SIS powder. Such processing can be carried out, for example, with a manual arbor press with a cylindrical brass ingot placed on top of the frozen specimens. The ingot serves as an interface between the specimens and the arbor of the press. Liquid nitrogen can be periodically added to the SIS specimens to keep them frozen.

Other methods for comminuting SIS specimens may be utilized to produce an SIS powder usable in accordance with the present invention. For example, SIS specimens can be freeze-dried and then ground using a manual arbor press or other, grinding means. Alternatively, SIS can be processed in a high shear blender to produce, upon dewatering and drying, an SIS powder.

Further grinding of the SIS powder using a pre-chilled mortar and pestle can be used to produce consistent, more finely divided product. Again, liquid nitrogen is used as needed to maintain solid frozen particles during final grinding. The powder can be easily hydrated using, for example, buffered saline to produce a fluidized tissue graft material at the desired viscosity.

### SIS Solutions

SIS powder is sifted through a wire mesh into any convenient vessel. The powder is then subjected to proteolytic digestion to form substantially homogeneous solutions. In one embodiment, the powder is digested with 1 mg/ml of pepsin (Sigma Chemical Co., St. Louis, Mo.,) in 0.1 M acetic acid, adjusted to pH 2.5 with hydrochloric acid, over a 48 hour period at room temperature. The reaction medium is neutralized with sodium hydroxide to inactivate the peptic activity. The solubilized submucosa may then be concentrated by salt precipitation of the solution and separated for further purification and/or freeze drying to form a protease solubilized intestinal submucosa in powder form

The viscosity of fluidized submucosa compositions can be manipulated by controlling the concentration of the submucosa component and the degree of hydration. The viscosity can be adjusted to a range of about 2 to about 300,000 cps at 25° C. Low viscosity submucosa compositions are better adapted for applications or applications within body cavities. Higher viscosity formulations, for example, gels, can be prepared from the SIS digest solutions by adjusting the pH of such solutions to about 6.0 to about 7.0.

SIS has also been described as being formed into a gel by mixing 0.1 N NaOH [ 1/10 of the volume of digest solution) and 10×PBS pH 7.4 ( 1/9 of the volume of digest solution) in appropriate amounts at 4° C. The solution was brought to the desired \volume and concentration using cold (40q 1×PBS pH 7.4 and placed in a 37° C. incubator for gelation to occur.

The ECM was able to form a matrix after 40 minutes in solution. The ECM-derived gel was liquid at temperatures below 200° C., but turned into a gel when the temperature was raised to 37° C.

In preparing gels from ECM, all of the solutions should be kept on ice and the following variables must be determined in accordance with U.S. Pat. No. 8,361,503, which is hereby incorporated by reference in its entirety:

Cf=concentration of the final gel in mg/ml

Cs=concentration of the ECM digest solution in mg/ml

Vf=volume of the final gel solution needed for the experiments

Vd=volume needed from the ECM digest solution in ml

V10x=volume of 10×PBS needed in ml

V1x=volume of 1×PBS needed in ml

VNaOH=volume of 0.1 N NaOH needed in ml

First, determine the final concentration (Cf) and volume (Vf) of ECM gel required. Then, calculate the mass of ECM needed by multiplying Cf (mg/ml)*Vf (ml). This value will provide the volume needed from the ECM digest solution (Vd) where Vd=[Cf (mg/ml)*Vf(ml]/Cs.

Calculate the volume of 10×PBS needed by dividing the calculated volume Vd by 9 (V10x=Vd/9). Calculate the volume of 0.1 N NaOH needed by dividing the calculated volume Vd by 10 (VNaOh=Vd/10). Calculate the amount of 1×PBS needed to bring the solution to the appropriate concentration/volume as follows: V1 x″ Vf-Vd-V10x-VNaOH. Add all the reagents (V1x+Vd+V10x.+VNaOH) to an appropriate container (usually 15 or 50 ml centrifuge tubes) without the ECM digest (Vd).

Place solutions on ice and keep on ice at all times. Add the appropriate volume from the ECM digest solution (Vd) to the PBS/NaOH mixture prepared above and mix well with a 1 ml micropipette while being careful to avoid creation of air bubbles in the solution. Depending on the viscosity of the ECM digest solution, there might be some significant volume loss during the transfer. Monitor the total volume and add appropriate amounts until the final volume is achieved. Measure the pH of the pre-gel solution—the pH should be around 7.4. Add the pre-gel solution to a mold or to appropriate wells. Place the mold or wells in a 37° C. incubator for a minimum of 40 minutes. Avoid using an incubator with carbon dioxide control. If water evaporation is a concern, place the mold inside a plastic sealable bag before placing in the incubator. After gelation, the gel can be removed from the mold and placed on 1×PBS. If the gels were made in tissue culture plates, 1×PBS can be placed on top of the gels until use to maintain the gels hydrated. Sample calculation: Make 6 ml of gel with a final concentration of 6 mg/ml from the 10 mg/ml stock solution.

### SIS-ECM Administration Procedure

SIS-ECM solution or suspension can be administered by enema into the colon of the UC-induced rats. No rejection, infection, or abnormal physiologic response of the host animal is expected following administration of the graft. The solution or suspension may also be administered via endoscopy and via laparoscopy into the colon. It is believed that an unexpected result of the current invention is the stimulation of appropriate tissue remodeling such that augmentation of colon mucosa can be accomplished with SIS solution or suspension material.

The fluidized compositions can result in tissue replacement and repair, and further result in treatment or cure of IBD, including UC. The fluidized submucosal compositions are used in accordance with the present method to induce regrowth of natural colon mucosal tissue. By injecting an effective amount of a fluidized ECM composition into or onto the locale of the defective tissue, the biological properties can be realized without the need for more invasive surgical techniques.

1-77. (canceled)

78. A method for treating the rectum or colon, comprising:
accessing rectal or colonic mucosa through a trans-anal approach;
performing a mucosectomy to remove mucosal tissue from at least a portion of a rectal or colonic wall; and
securing a flexible scaffold material to a region of the rectal colonic wall from which mucosa has been removed, wherein the scaffold material is selected to facilitate cellular ingrowth and formation of new mucosal tissue.

79. The method of claim 78, wherein the mucosectomy is performed over substantially the entire surface of the rectal mucosa.

80. The method of claim 78, wherein the mucosectomy is performed over substantially the entire surface of the rectal mucosa and at least a portion of the sigmoid colon.

81. The method of claim 78, wherein the scaffold comprises an extracellular tissue matrix.

82. The method of claim 81, wherein the extracellular tissue matrix has been processed to remove substantially all cells from the tissue matrix.

83. The method of claim 81, wherein the extracellular tissue matrix comprise a small intestine submucosal tissue matrix.

84. The method of claim 81, wherein the extracellular tissue matrix is derived from a tissue selected from at least one of tendon, urinary bladder, skeletal muscle, esophagus, large intestine, small intestine, gastric tissue, pericardial tissue, dermis, ligament, muscle, adipose tissue, and fascia.

85. The method of claim 78, wherein the scaffold is in the form of one or more layers of flexible sheets.

86. The method of claim 85, wherein securing a flexible scaffold material to a region of the colonic wall from which mucosa has been removed comprises securing multiple layers of flexible sheets to the rectal or colonic wall.

87. The method of claim 86, wherein the multiple layers comprise at least four layers.

88. The method of claim 86, wherein the multiple layers comprise at least eight layers.

89. The method of claim 78, wherein the scaffold comprises an elastic material.

90. The method of claim 89, wherein the material has sufficient elasticity to allow normal colonic peristalsis.

91. The method of claim 78, wherein the scaffold comprises at least one synthetic material.

92. The method of claim 91, wherein the synthetic material comprises poly(ester urethane)urea.

93. The method of claim 91, wherein the scaffold comprises an extracellular matrix component and a synthetic component.

94. The method of claim 78, wherein securing a flexible scaffold material to a region of the rectal or colonic wall from which mucosa has been removed comprises suturing the scaffold material to the rectal or colonic wall.

95. The method of claim 78, wherein securing a flexible scaffold material to a region of the rectal or colonic wall from which mucosa has been removed comprises stapling the scaffold material to the rectal or colonic wall.

96. The method of claim 78, wherein securing a flexible scaffold material to a region of the rectal or colonic wall from which mucosa has been removed comprises clipping the scaffold material to the rectal or colonic wall.

97. The method of claim 78, wherein securing a flexible scaffold material to a region of the rectal or colonic wall from which mucosa has been removed comprises applying an adhesive to the scaffold material and the rectal or colonic wall.

98. The method of claim 78, wherein securing a flexible scaffold material to a region of the rectal or colonic wall from which mucosa has been removed comprises positioning a stent within the rectum or colon.

99. The method of claim 98, wherein the stent comprises a self-expanding stent.

100. The method of claim 78, further comprising performing endoscopic mucosal resection on a portion of the colon.

101. The method of claim 100, further comprising securing an additional flexible scaffold material to a region of the rectal or colonic wall from which mucosa has been removed using endoscopic mucosal resection.

102. The method of claim 78, wherein the flexible scaffold materials are in the form of a cylinder.

103. The method of claim 78, wherein the flexible scaffold materials comprise a meshed sheet.

104. The method of claim 78, wherein the flexible scaffold materials are in the form of a cylinder wound from a strip of material.

105. The method of claim 78, wherein the mucosectomy is performed on tissue affected by inflammatory bowel disease.

106. The method of claim 105, wherein the mucosectomy is performed on tissue affected by ulcerative colitis.

107. The method of claim 78, wherein the scaffold comprises at least one therapeutic agent.

108. The method of claim 107, wherein the therapeutic agent is selected from antibiotics, antimicrobials, antifungals, anti-inflammatories, tissue growth factors, and ionic silver.

109. A method for treating the rectum or colon, comprising:
performing a mucosectomy to remove mucosal tissue from at least a portion of a rectal or colonic wall; and
securing a flexible and elastic scaffold material to a region of the rectal or colonic wall from which mucosa has been removed, wherein the scaffold material is selected to facilitate cellular ingrowth and formation of new mucosal tissue.

110. The method of claim 109, wherein the mucosectomy is performed over substantially the entire surface of the rectal mucosa.

111. The method of claim 109, wherein the mucosectomy is performed over substantially the entire surface of the rectal mucosa and at least a portion of the signmoid colon.

112. The method of claim 109, wherein the scaffold comprises an extracellular tissue matrix.

113. The method of claim 112, wherein the extracellular tissue matrix has been processed to remove substantially all cells from the tissue matrix.

114. The method of claim 112, wherein the extracellular tissue matrix comprise a small intestine submucosal tissue matrix.

115. The method of claim 112, wherein the extracellular tissue matrix is derived from a tissue selected from at least one of tendon, urinary bladder, skeletal muscle, esophagus, large intestine, small intestine, gastric tissue, pericardial tissue, dermis, ligament, muscle, adipose tissue, and fascia.

116. The method of claim 109, wherein the scaffold is in the form of one or more layers of flexible sheets.

117. The method of claim 116, wherein securing a flexible scaffold material to a region of the rectal or colonic wall from which mucosa has been removed comprises securing multiple layers of flexible sheets to the rectal or colonic wall.

118. The method of claim 117, wherein the multiple layers comprise at least four layers.

119. The method of claim 118, wherein the multiple layers comprise at least eight layers.

120. The method of claim 109, wherein the material has sufficient elasticity to allow normal colonic peristalsis.

121. The method of claim 109, wherein the scaffold comprises at least one synthetic material.

122. The method of claim 121, wherein the synthetic material comprises poly(ester urethane)urea.

123. The method of claim 121, wherein the scaffold comprises an extracellular matrix component and a synthetic component.

124. The method of claim 109, wherein securing a flexible scaffold material to a region of the rectal or colonic wall from which mucosa has been removed comprises suturing the scaffold material to the rectal or colonic wall.

125. The method of claim 109, wherein securing a flexible scaffold material to a region of the rectal or colonic wall from which mucosa has been removed comprises stapling the scaffold material to the rectal or colonic wall.

126. The method of claim 109, wherein securing a flexible scaffold material to a region of the rectal or colonic wall from which mucosa has been removed comprises clipping the scaffold material to the rectal or colonic wall.

127. The method of claim 109, wherein securing a flexible scaffold material to a region of the rectal or colonic wall from which mucosa has been removed comprises applying an adhesive to the scaffold material and the rectal or colonic wall.

128. The method of claim 109, wherein securing a flexible scaffold material to a region of the rectal or colonic wall from which mucosa has been removed comprises positioning a stent within the rectum or colon.

129. The method of claim 128, wherein the stent comprises a self-expanding stent.

130. The method of claim 109, further comprising performing endoscopic mucosal resection on a portion of the colon.

131. The method of claim 130, further comprising securing an additional flexible scaffold material to a region of the colonic wall from which mucosa has been removed using endoscopic mucosal resection.

132. The method of claim 109, wherein the flexible scaffold materials are in the form of a cylinder.

133. The method of claim 109, wherein the flexible scaffold materials comprise a meshed sheet.

134. The method of claim 109, wherein the flexible scaffold materials are in the form of a cylinder wound from a strip of material.

135. The method of claim 109, wherein the mucosectomy is performed on tissue affected by inflammatory bowel disease.

136. The method of claim 135, wherein the mucosectomy is performed on tissue affected by ulcerative colitis.

137. The method of claim 109, wherein the scaffold comprises at least one therapeutic agent.

138. The method of claim 137, wherein the therapeutic agent is selected from antibiotics, antimicrobials, antifungals, anti-inflammatories, tissue growth factors, and ionic silver.

139. A device for treating diseases of the rectum or colon, comprising:
a porous scaffold having a tubular shape, wherein the scaffold material is selected to facilitate cellular ingrowth and formation of new mucosal tissue, and wherein the scaffold has sufficient elasticity and flexibility to withstand forces exerted by rectal or colonic peristalsis when implanted in the rectum or colon.

140. The device of claim 139, wherein the scaffold comprises an extracellular tissue matrix.

141. The device of claim 140, wherein the extracellular tissue matrix has been processed to remove substantially all cells from the tissue matrix.

142. The device of claim 140, wherein the extracellular tissue matrix comprise a small intestine submucosal tissue matrix.

143. The device of claim 112, wherein the extracellular tissue matrix is derived from a tissue selected from at least one of tendon, urinary bladder, skeletal muscle, esophagus, large intestine, small intestine, gastric tissue, pericardial tissue, dermis, ligament, muscle, adipose tissue, and fascia.

144. The device of claim 139, wherein the scaffold comprises one or more layers of flexible sheets.

145. The device of claim 144, wherein the multiple layers comprise at least four layers.

146. The device of claim 144, wherein the multiple layers comprise at least eight layers.

147. The device of claim 139, wherein the scaffold comprises at least one synthetic material.

148. The device of claim 147, wherein the synthetic material comprises poly(ester urethane)urea.

149. The device of claim 147, wherein the scaffold comprises an extracellular matrix component and a synthetic component.

150. The device of claim 139, further comprising a stent for securing the scaffold to a rectal or colonic wall.

151. The device of claim 150, wherein the stent comprises a self-expanding stent.

152. The device of claim 139, wherein the flexible scaffold materials comprise a meshed sheet.

153. The device of claim 139, wherein the flexible scaffold materials are in the form of a cylinder wound from a strip of material.
